(19) United States
(12) Patent Application Publication
(10) Pub. No.: US 2015/0018833 A1
(43) Pub. Date: Jan. 15, 2015

(54) Cup Impactor

(71) Applicants: Jonathan P. Crandall, Fort Wayne, IN (US); Douglas J. Roger, Rancho Mirage, CA (US)

(72) Inventors: Jonathan P. Crandall, Fort Wayne, IN (US); Douglas J. Roger, Rancho Mirage, CA (US)

(73) Assignee: Greatbatch Ltd., Clarence, NY (US)

(21) Appl. No.: 13/198,060

(22) Filed: Aug. 4, 2011

Related U.S. Application Data
(60) Provisional application No. 61/370,487, filed on Aug. 4, 2010.

(51) Int. Cl. A61B 17/56

(52) U.S. Cl. 606/91

(57) ABSTRACT

An orthopedic cup impactor for use in minimally invasive hip replacement surgical procedures is described. The impactor comprises a handle, residing at a proximal end portion, and a cup engagement sub-assembly located at a distal portion. A shaft resides therebetween. The shaft portion is designed with a large radius of curvature that provides added clearance when inserting the impactor in obese patients. The cup engagement sub-assembly features a drive train that comprises a series of “U” and “H” joints deigned to provide full rotational motion. The drive train may be designed to be removable from the cup impactor to provide more efficient and thorough cleaning.

### CROSS-REFERENCE TO RELATED APPLICATIONS

This application claims priority from U.S. Provisional Patent Application Ser. No. 61/370,487, filed Aug. 4, 2010.

### FIELD OF THE INVENTION

The present invention is directed to minimally invasive surgery, and more particularly to an acetabular prosthetic cup impactor tool for use in minimally invasive hip replacement surgery.

### PRIOR ART

Approximately 200,000 hip replacements are performed each year in the United States and the number is expected to continue to grow as the population continues to age. The usual reasons for hip replacement are osteoarthritis, rheumatoid arthritis and traumatic arthritis, all of which can cause pain and stiffness that limit mobility and the ability to perform daily living activities. Hip replacement surgery is usually performed where other measures (e.g. physical therapy, medications, and walking aids) are unable to overcome the chronic pain and disability associated with these conditions.

Obesity is an increasingly common health concern in the United States. According to the Center for Disease Control and Prevention (CDC), about one third of the U.S. population is obese. Studies have suggested that obesity is linked to the development of joint ailments, particularly of the hip and knee. These studies disclosed, for example, that obesity increases the risk for developing osteoarthritis in the hip and the knee, and suggest that obesity plays a role in initiating and accelerating hip and knee osteoarthritis. The development of osteoarthritis occurs either directly by the increased load on a joint or indirectly because obesity is associated with a variety of metabolic disorders. Additionally, the added weight of an obese person contributes to the stresses that are applied to a person's joints thereby increasing joint wear, and in so doing accelerating the need for replacement. Therefore, there is an increasing need to address joint ailments for obese patients as well.

Various techniques are used by orthopedic surgeons to perform hip replacements. These include the following approaches: anterior, antero-lateral, lateral, postero-lateral and posterior. The posterior and postero-lateral approaches account for approximately 60%-70% of hip replacement surgeries.

Traditional hip replacement surgery involves an open surgical procedure and extensive surgical dissection. However, such procedures require a longer recovery period and rehabilitation time for the patient. The average hospital stay for open hip replacement procedures is 4-5 days, followed in most cases by extensive rehabilitation.

More recently, there has been considerable interest and research done in Minimally invasive Surgery (MIS), including the use of MIS procedures in connection with hip replacement surgery. In comparison with the traditional open surgical approach, MIS hip replacement surgeries involve fewer traumas to the muscles surrounding the hip joint. Specifically, fewer muscles that help to stabilize the hip joint are cut in MIS hip replacement surgeries, reducing the risk of dislocation of the hip surgery and speeding recovery. Patients spend less time in the hospital and return to normal life activities more quickly.

MIS approaches use smaller surgical openings, which require specialized instruments to perform hip replacement procedures. As such, these MIS procedures are beneficial since they are less traumatic to the body. However, these MIS procedures are particularly difficult to perform with obese patients. The increased body mass and overall tissue volume of obese patients add additional complications in performing MIS procedures, particularly in accessing the surgical site.

In these cases, the incision is especially deep as there are thicker and deeper masses of soft tissue. Traditional acetabular cup impactors provide some clearance of soft tissue. However, traditional impactors provide inadequate clearance particularly when performing a MIS procedure on an obese patient. Accordingly, there is a need for an improved impactor tool for use in MIS orthopedic procedures (e.g., hip replacement surgery) with obese patients that addresses some of the shortcomings in the existing surgical impactors noted above.

### SUMMARY OF THE INVENTION

In accordance with one embodiment, an orthopedic cup impactor for use in minimally invasive hip replacement surgical procedures is provided. The impactor comprises a handle, residing at a proximal end portion and a cup engagement sub-assembly located at a distal portion. A shaft resides therebetween. The shaft portion is designed with a large radius of curvature that provides added clearance when inserting the impactor in obese patients. The shaft portion is further designed with a curved underside surface and a planar top surface with beveled side edges. These features aid in the insertion of the impactor and provide surfaces to aid in the leverage of the tissue.

In accordance with another embodiment, the impactor of the present invention features an offset between the handle portion and the distal portion. The offset between the handle portion and the distal end allows for a much deeper insertion of the cup impactor into obese patients than traditional impactors with obese patients.

In accordance with an additional embodiment, the impactor of the present invention features a shaft with a curved cross-section. This feature enables the impactor access into an obese patient with increased efficiency. Furthermore, the shaft's curved cross-section helps to retard tissue necrosis.

In accordance with yet another embodiment, the impactor features a cup engagement subassembly comprising a drive shaft having multiple degrees of freedom. This drive shaft design feature, comprising a series of “U” and “H” joints, provides full rotation at differing bend angles when inserting an orthopedic implant. Furthermore, in yet another embodiment, the drive train may be designed to be removable from the cup impactor. Such a feature allows for efficient and thorough cleaning of the drive train after a surgical procedure.

### BRIEF DESCRIPTION OF THE DRAWINGS

FIG. 1 is a perspective view of an embodiment of a cup impactor.

FIG. 2 is a side view illustrating alternate orientations of the cup impactor embodiment shown in FIG. 1.

FIG. 3 illustrates a bottom view of the embodiment of the cup impactor shown in FIG. 1.

FIG. 4 shows a magnified cross-sectional view of a distal end portion of the cup impactor embodiment shown in FIG. 1.

FIG. 5 illustrates a side view of an embodiment of a drive train of the present invention.

FIG. 6 shows a magnified side view of the embodiment of the drive train shown in FIG. 5.

FIG. 7 is a perspective view of the cup impactor with an embodiment of a drive tool.

FIG. 8 shows an end view of the cup impactor embodiment shown in FIG. 1.

FIG. 8A illustrates an alternate embodiment of the cup impactor shown in FIG. 1.

### DETAILED DESCRIPTION OF THE PREFERRED EMBODIMENTS

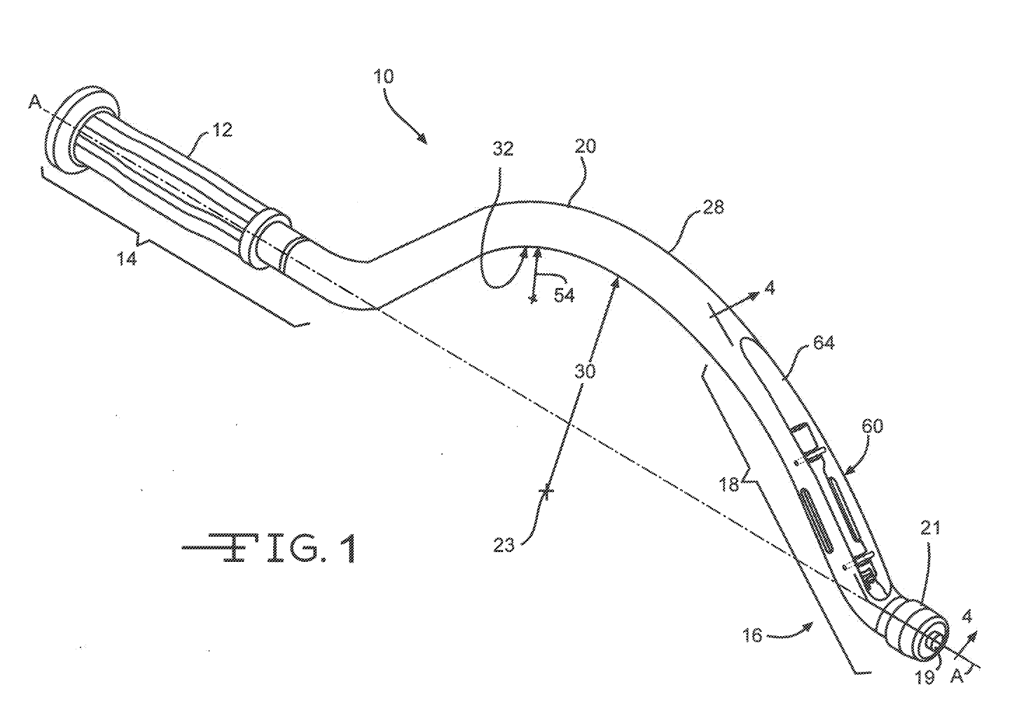

Now turning to the figures, FIGS. 1 to 8A illustrate embodiments of a cup impactor 10 of the present invention. As illustrated in FIG. I, the cup impactor 10 comprises a handle 12 located at a proximal end portion 14 and an orthopedic cup engagement sub-assembly 16 located at a distal end portion 18 thereof. A shaft 20 resides between the respective handle portion 12 and the distal end portion 18 of the impactor 10.

The cup impactor 10 has an impactor length 22 and an impactor height 24 (FIG. 7). In a preferred embodiment, the impactor length 22 ranges from about 20 cm to about 100 cm, more preferably, from about 40 cm to about 60 cm. In a further embodiment, the impactor height 24 ranges from about 5 cm to about 20 cm, more preferably, from about 10 cm to about 15 cm.

In an embodiment, the shaft 20 is curved between the handle 12 residing at the proximal end 14 and the distal end 19 of the impactor 10. Preferably, the shaft 20 is curved similarly to that of an arc 26 with an apex 28 positioned at about its maximum height. In a preferred embodiment, the arc 26 has a radius of curvature 30 that ranges from about 10 cm to about 20 cm as measured with respect to an inner surface 32 thereof.

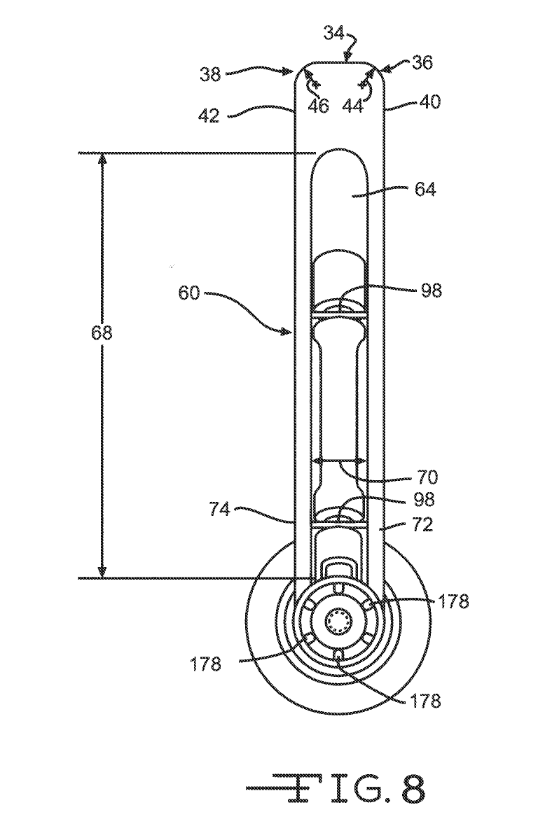

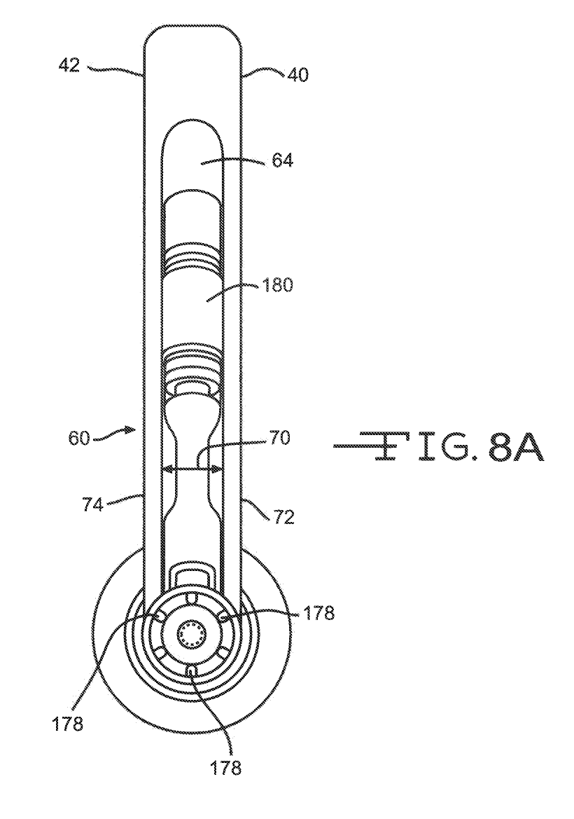

The shaft 20 preferably has a planar top surface 34. Beveled top side edges 36, 38 transition from the top surface 34 to respective left and right sidewalls 40, 42 of the shaft 20 (FIG. 8). The beveled side edges 36, 38 further extend longitudinally from the handle portion 12 to the distal end 19 of the impactor 10. In a preferred embodiment, the top side edges 36, 38 have a radius of curvature 44, 46 that ranges from about 0.1 cm to about 2 cm. Furthermore, the left and right sidewalls 40, 42 may have a surface 48 that is planar. Alternatively, the left or right sidewall 40, 42 may also have a curved surface 48.

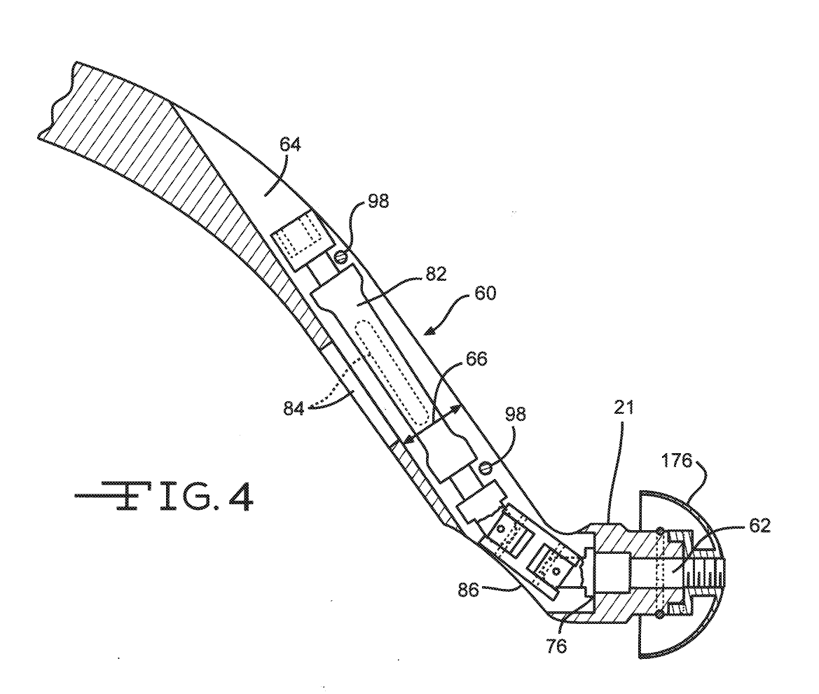

In a preferred embodiment, the inner surface 32 has curved inner surface side edges 50, 52. These side edges 50, 52 are designed such that they fluidly transition into the left and right sidewalls 40, 42 of the shaft 20, as illustrated in FIG. 3. In a preferred embodiment, the inner surface 32 has an inner surface radius of curvature 54 that ranges from about 1 cm to about 5 cm.

Alternatively, the shaft 20 could be constructed such that it has a curved cross-section and more preferably, a round cross-section. As such, the shaft 20 may have a diameter that ranges from about 1 cm to about 10 cm. The curved cross-section of the shaft 20 is beneficial because it reduces the physical resistance of the cup impactor 10 as it is inserted within the body of a patient. Reduced resistance is especially beneficial when the impactor 10 is inserted within an obese human body of a large mass and volume. The curved surfaces of the impactor 10 allow the user to turn and rotate the instrument more efficiently. Furthermore, the arc design of the shaft 20 provides for improved access to the hip area of the patient.

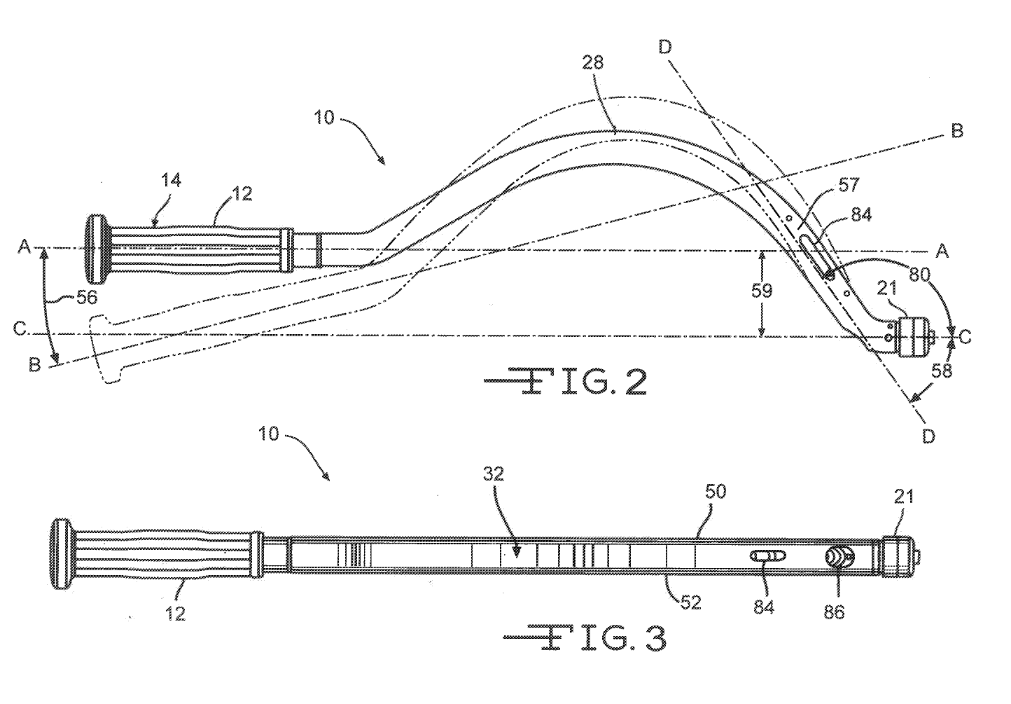

In a preferred embodiment, the handle 12 is positioned such that it is about coplanar with that of the distal end 19 of the impactor 10. As illustrated in FIG. 1, longitudinal axis A-A extends through the center of the handle 12 and through the distal end 19 of the impactor 10. Alternatively, as shown in FIG. 2, the handle 12 may be positioned such that it is offset from the plane of the distal end 19, i.e., deviating from axis A-A. In a further embodiment, a handle offset angle 56 is established between longitudinal axis A-A and handle axis B-B. Axis B-B is herein defined as the axis that extends longitudinally through the center of the handle portion 12. Handle axis B-B can therefore assume multiple positions depending on the particular handle offset that is desired, as shown in FIG. 2. The handle offset angle 56 is herein defined as the angle 56 between the intersection of longitudinal axis A-A and handle axis B-B. It is preferred that the handle offset angle 56 range from about 2° to about 40°.

Furthermore, the distal end 19 of the impactor 10 may be constructed such that it is offset from longitudinal axis A-A. In an additional embodiment, a distal end offset angle 58 is established between axis C-C, an axis extending longitudinally through the center of the distal end 19 of the impactor 10, and imaginary line D-D (FIG. 2). Line D-D is an imaginary line that extends about the middle of a distal portion 57 of the shaft 20, along sidewall 40, 42 as shown in FIG. 2. The distal end offset angle 58 is herein defined as the angle between the intersection of axis C-C and imaginary line D-D. It is further preferred that the distal end offset angle 58 may range from about 40° to about 60°. Additionally, the offset of the distal end 19 from the proximal end 14 may be defined by a distal end offset distance 59. The distal end offset distance 59 is herein defined as the distance between longitudinal axis A-A and axis CC as shown in FIG. 2. In a preferred embodiment, the offset distance 59 ranges from about 1 cm to about 10 cm.

It is further contemplated that the cup impactor 10 may or may not have an offset handle angle 56 or a distal end offset angle 58 or a distal end offset distance 59. Furthermore, the respective offset angles 56, 58 of the impactor 10 may be offset at angles that are similar or different from each other.

The cup engagement sub-assembly 16 comprises a drive train 60 that extends to a rod end 62 as shown in FIGS. 1, 4, and 7-8. The cup engagement sub-assembly 16 preferably resides at the distal end portion 18 of the impactor 10. In a preferred embodiment, the drive train 60 at least partially resides within a cavity 64 at the distal end portion 18 of the impactor 10.

The cavity preferably extends from the distal end 19 of the impactor 10 to a region proximate the distal end 19. The cavity 64 preferably furthermore resides within the top surface 34 of the shaft 20 of the impactor 10. In a preferred embodiment, the cavity 64 has a cavity depth 66 from about 1 cm to about 4 cm, a cavity length 68 from about 10 cm to about 20 cm and a cavity width 70 from about 1 cm to about 5 cm. Left and right cavity sidewalls 72, 74 extend along the length 68 of the cavity 64. The cavity 64 is further positioned such that it extends through the distal end 19 of the impactor 10 creating an opening 76 thereof. The opening 76 is preferably dimensioned such that at least a portion of the distal end of the cup engagement sub-assembly 16, particularly the rod 62 of the sub-assembly 16, extends therethrough. In a preferred embodiment, the opening 76 at the distal end 19 may have a diameter that ranges from about 0.5 cm to about 2 cm. In a preferred embodiment, the cavity 64 ends at a position that is distal of the apex 28 of the middle shaft portion and provides for receiving a driver tool 78 for rotating the drive shaft with the threaded rod 62 being at an angle 80 from about 40° to about 60°, preferably at about 55° with respect to a major shaft 82 of the drive train 60.

Furthermore, the depth 66 of the cavity 64 may be designed such that it gradually increases from the proximal end 18 to the distal end 19 of the impactor 10. The maximum cavity depth 66 is achieved at the opening 76 of the distal end 19 of the impactor 10. This design feature of the cavity 64 allows for improved unobstructed motion of the drive train 60 within the cavity 64 and provides an improved means of accessing the drive train 60 within the body of the patient.

The cavity 64 further has a series of slots 84 that extend through each of the cavity sidewalls 72, 74 and bottom surface 32 of the shaft 20. These slots 84 are designed to allow for efficient and thorough cleaning of the cavity 64. Furthermore, the cavity 64 has an additional opening 86 extending through the inner surface 32 of the shaft 20 distal of the slot openings 84. This additional opening 86 is preferably positioned along a bend 88 where the distal end 19 transitions into the arc 28 of the shaft 20. The opening 86 provides for easy access to the cup engagement sub-assembly 16 to allow for efficient and through cleaning thereof.

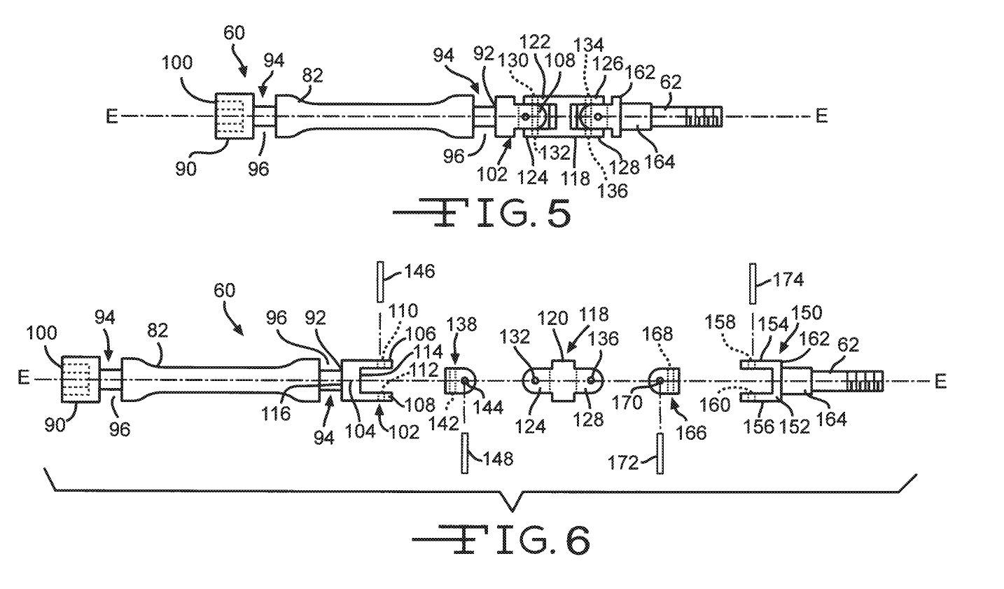

As particularly shown in FIGS. 4-6, the drive train 60 comprises a major shaft 82 as a cylindrically-shaped member having a proximal portion 90 and a distal end 92 with a length therebetween. Furthermore, the major shaft 82 comprises a bar bell portion 94 at both the proximal 90 and distal ends 92 thereof. The bar bell portion 94 is designed such that a portion of the major shaft 82 is removed to create a recessed region 96 along the shaft 82. This recessed shaft region 96 is characterized with a shaft diameter that is smaller than that of the major shaft 82. The recessed shaft region 96 is designed such that it enables a pin or pins 98 to be positioned across the region 96 between the cavity sidewalls 72, 74 thereby permitting rotational movement of the shaft while preventing the drive shaft 82 from being removed from the cavity 64 of the impactor 10, as shown in FIGS. 1 and 4.

The proximal shaft end 90 preferably has a socket 100 therewithin designed to engage the drive tool 78 (FIG. 6). The drive tool 78 is designed to be inserted into the socket 100 of the proximal end 90 of the drive train 60. The drive tool 78 comprises a hexagonal end or similar type structure that provides flats for detachable connection of the socket 100 at the proximal end 90. In a preferred embodiment, the drive shaft 60 may be rotated clockwise or counterclockwise when the tool 78 is engaged in the socket 100. Rotation of the drive shaft 82 in turn rotates the threaded rod 62 at the distal end 19 of the impactor 10. Alternately, a rotary drive power source (not shown) could also engage the socket 100 of the drive shaft 82 to provide rotation.

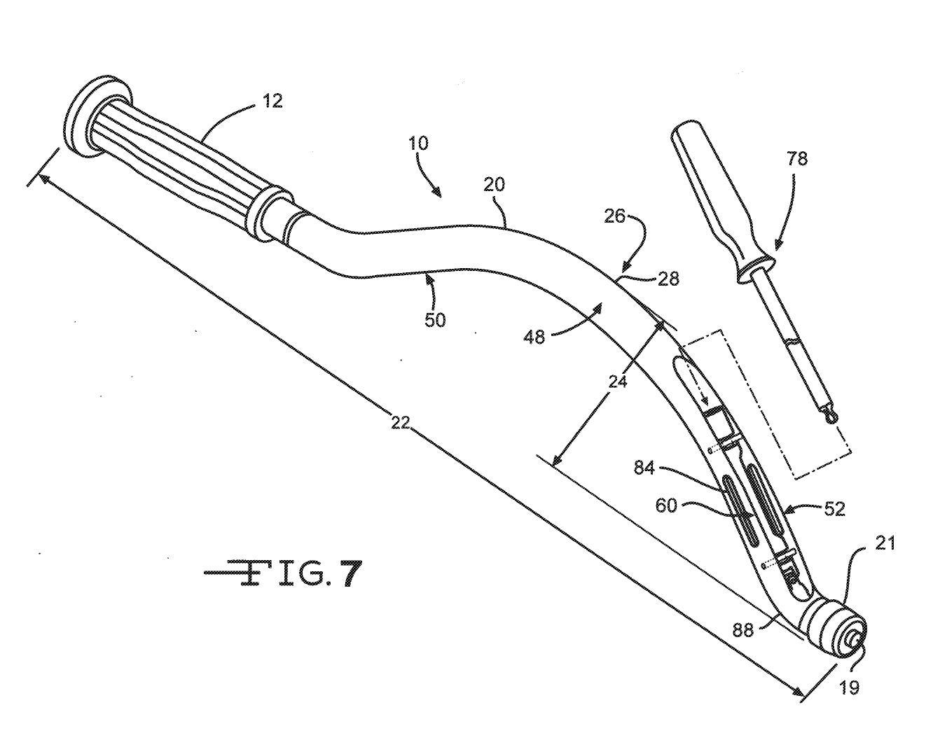

As particularly shown in FIGS. 5 and 6, a first or proximal U-joint 102 is supported at the distal end 92 of the major shaft 82. The proximal U-joint 102 is comprised of a proximal cylindrical sidewall 104 supporting a pair of yoke plates 106 and 108 having respective openings 110, 112. Connection of the U-joint 102 to the shaft 82 may be made by a screw and the like. The screw is received in an opening 114 in the sidewall 104 and seats against a flat 116 at the distal shaft end 92. In the alternative, the proximal U-joint could be welded or otherwise secured in place or, the U-joint and shaft could be machined from a single piece of material.

The drive train 60 further includes an H-shaped joint 118 comprising a cylindrical intermediate section 120 supporting opposed first and second pairs of yoke plates 122, 124 and 126, 128. Respective openings 130, 132 and 134, 136 are provided in the yoke plates. A proximal pivot block 138 (FIG. 6) resides between the yoke plates 106, 108 of the proximal U-joint 102 and the first pair of yoke plates 122, 124 of the H-joint 118. The proximal pivot block 138 comprises two pairs of perpendicularly opposed openings 142 and 144.

Pin 146 is received in the openings 110, 112 in the yoke plates 106 and 108 of the U-joint 102 and the opening 142 in the pivot block 138, and a pin 148 is received in the opening 142 of the pivot block 138 and the openings 110, 112 of the yoke plates 122, 124 of the H-plate 118 to thereby pivotably secure the proximal U-joint 102 to the first end of the H-joint 118. It is noted that only one of the pins 146 or 148 extends completely from one face of the pivot block 138 to the other face. As passage from one face to the other is blocked by the first pin, the other of the two pins 146 or 148 is two “half pins”.

As shown in FIGS. 5 and 6, the drive train 60 also includes a distal U-joint 150 that comprises a distal cylindrical side wall 152 supporting a pair of yoke plates 154 and 156 having respective openings 158, 160. Opposite the yoke plates, the cylindrical sidewall 152 meets a base plate 162 having an enlarged diameter. A cylinder 164 extends outwardly from the base plate 162. The threaded rod 62 preferably extends outwardly from the cylinder 164 of the distal U-joint 150. Each of the components of the drive train 60 have their respective axes aligned parallel to each other and co-axial with, but spaced from, a longitudinal axis E-E of the distal U-joint 150.

A distal pivot block 166, similar in structure to the proximal pivot block 138, comprises two pairs of perpendicularly opposed openings 168 and 170. Pin 174 is received in the openings 158, 160 in the respective yoke plates 154, 156 of the distal U-joint 150 and the opening 168 in the pivot block 166, and a pin 172 is received in the openings 134, 136 of the respective yoke plates 126, 128 of the H-joint 118 and opening 168 of the pivot block 166 to thereby pivotably secure the distal U-joint 150 to the second or distal end of the H-joint 118. As with the pivotable connection between the H-joint 118 and the proximal U-joint 102, only one of the pins 172, 174 extends the full width of the pivot block 166 from one face to an opposite face thereof. The other pin is provided as two partial length pins.

In this manner, the drive train 60 comprising the drive shaft 82, the proximal U-joint 102, the first pivot block 138, the H-joint 118, the second pivot block 166 and the distal U-joint 150 provides for transmission of rotational motion imparted to the proximal end of the shaft 82 to the base plate 162 and its supported rod 62.

Although the H-joint 118 is preferred, it is contemplated that the drive train 60 may be constructed without the H-joint 118. In this embodiment, the drive train 60 would comprise the drive shaft 82, the proximal U-joint 102, the first pivot block 138 and the distal U-joint 150. It is further contemplated that the drive train 60 may comprise a flexible shaft design such as wire wound shaft or a shaft that is laser cut.

The threaded rod 62 extends through the distal end 19 of the impactor 10. The threaded rod 62 preferably engages with an orthopedic implant 176. Prior to the surgical procedure, a connection between the threaded rod 62 and the orthopedic implant 176 is established. In a preferred embodiment, the threaded rod 62 is mated with corresponding grooves (not shown) of the implant 176. Once the implant is secured to the distal end 19 of the impactor 10, the implant 176 is inserted into a patient. Once the implant 176 is correctly positioned within the body, the drive shaft 82 is rotated in a reverse direction with respect to the threads of the rod 62. Typically, the rod 62 is provided with right hand threads so that counterclockwise rotation disengages the implant 176 from the impactor 10.

Additionally, a series of slots 178, as shown in FIG. 7, may extend around the perimeter of the base plate 162 of the threaded rod 62. These slots 178 are designed such that they fit with corresponding implant slot grooves (not shown) providing additional support between the threaded rod 62 of the impactor 10 and the implant 176.

The drive train 60 may be designed without pins 98 such that it is removable from the cavity 64 of the impactor 10. A removable drive shaft 82 is beneficial in that it provides for more efficient and thorough cleaning of the cup engagement sub-assembly 16. As shown in an alternate embodiment of FIG. 8A, a sleeve 180 may be positioned over the proximal end portion 90 of the drive shaft 82. This sleeve 180 provides for a wider perimeter diameter that creates a snug or interference fit when positioned within the cavity 64. Alternatively the proximal end 90 of the drive shaft 82 may be constructed with an increased diameter that creates an interference fit when positioned between the sidewalls 72, 74 of the cavity 64.

Furthermore, it is contemplated that a plurality of pads may be positioned around the perimeter of the major shaft 82 of the drive train 60 and/or positioned along the inside surface of the cavity sidewalls 72, 74. These pads are designed to provide an additional interference fit within the cavity 64 such that the drive train 60 remains within the cavity 64 during the surgical procedure.

Of course, the forgoing description is that of certain features, aspects and advantages of the present invention, to which various changes and modifications can be made without departing from the spirit and scope of the present invention. Moreover, the cup impactors need not feature all of the objects, advantages, features and aspects discussed above. Thus, for example, those of skill in the art will recognize that the invention can be embodied or carried out in a manner that achieves or optimizes one advantage or a group of advantages as taught herein without necessarily achieving other objects or advantages as may be taught or suggested herein. In addition, while a number of variations of the invention have been shown and described in detail, other modifications and methods of use, which are within the scope of this invention, will be readily apparent to those of skill in the art based upon this disclosure. It is contemplated that various combinations or sub-combinations of these specific features and aspects of embodiments may be made and still fall within the scope of the invention. Accordingly, it should be understood that various features and aspects of embodiments may be made and still fall within the scope of the invention. Accordingly, it should be understood that various features and aspects of the disclosed embodiments can be combined with or substituted for one another in order to form varying modes of the discussed cup impactor embodiments.

1. An orthopedic cup impactor, which comprises:
a) a handle extending along a handle longitudinal axis from a proximal handle end to a distal handle end;
b) a curved shaft extending from a proximal curved shaft end to a distal curved shaft end, wherein the proximal curved shaft end is connected to the distal handle end;
c) a cup engagement portion extending along a cup engagement portion longitudinal axis from a distal end of the cup engagement portion to a proximal end of the cup engagement portion connected to the distal curved shaft end, wherein an apex of the curved shaft is intermediate where the proximal curved shaft end is connected to the distal handle end and where the distal curved shaft end is connected to the proximal end of the cup engagement portion;
d) a drive train at least partially disposed within the curved shaft and within the cup engagement portion, the drive train comprising a proximal drive train end extending to a distal drive train end;
e) wherein the cup engagement longitudinal axis is parallel to the handle longitudinal axis, and
f) wherein an imaginary line extending perpendicularly from the cup engagement portion longitudinal axis intersects the handle longitudinal axis and then the apex such that the curved shaft apex is a greater distance from the cup engagement longitudinal axis than from the handle longitudinal axis, and
g) wherein the proximal drive train end is adapted for releasable connection to a source of rotary motion so that rotational movement imparted to the drive train in a first direction causes the distal drive train end to engage with an implant and rotational movement imparted to the drive train in a second, opposite direction causes the distal drive train end to disengage from the implant.

2. (canceled)

3. The orthopedic cup impactor of claim 1, wherein the curved shaft comprises a planar top surface.

4. The orthopedic cup impactor of claim 3, wherein the top surface of the curved shaft has a beveled side edge with a radius of curvature from 0.1 cm to 2 cm.

5. The orthopedic cup impactor of claim 1, wherein an inner shaft sidewall surface of the curved shaft has a radius of curvature from 1 cm to 5 cm.

6. (canceled)

7. The orthopedic cup impactor of claim 1, wherein an offset radius of curvature of the curved shaft ranges from 10 cm to 20 cm.

8. The orthopedic cup impactor of claim 1, wherein the handle is co-planar with the distal end of the cup engagement portion.

9. (canceled)

10. (canceled)

11. The orthopedic cup impactor of claim 1, wherein the cup engagement longitudinal axis is offset with respect to the handle longitudinal axis by an offset distance from 1 cm to 10 cm.

12. The orthopedic cup impactor of claim 1, wherein a threaded rod at a distal portion of the drive train is at an angle from 120° to 140° with respect to an axis D-D that extends lengthwise through a major drive rod of the drive train.

13.-15. (canceled)

16. An orthopedic cup impactor, which comprises:
a) a handle extending along a handle longitudinal axis from a proximal handle end to a distal handle end;
b) a curved shaft extending from a proximal curved shaft end to a distal curved shaft end, wherein the proximal curved shaft end is connected to the distal handle end;
c) a cup engagement portion extending along a cup engagement portion longitudinal axis from a distal end of the cup engagement portion to a proximal end of the cup engagement portion connected to the distal curved shaft end, wherein an apex of the curved shaft is intermediate where the proximal curved shaft end is connected to the distal handle end and where the distal curved shaft end is connected to the proximal end of the cup engagement portion;
d) a drive train at least partially disposed within the curved shaft and within the cup engagement portion, the drive train comprising:
i) a drive rod extending from a proximal drive rod end to a distal drive rod end;
ii) a proximal U-joint connected to the distal drive rod end;
iii) a distal U-joint rotationally connected to the proximal U-joint; and
iv) a threaded rod extending distally from the distal U-joint for releasable connection to an implant; and
e) wherein the cup engagement longitudinal axis is parallel to the handle longitudinal axis, and
f) wherein an imaginary line extending perpendicularly from the cup engagement portion longitudinal axis intersects the handle longitudinal axis and then the apex such that the curved shaft apex is a greater distance from the cup engagement longitudinal axis than from the handle longitudinal axis, and
g) wherein the proximal drive rod end is adapted for releasable connection to a source of rotary motion so that rotational movement imparted to the drive train in a first direction causes the threaded rod to engage with an implant and rotational movement imparted to the drive train in a second, opposite direction causes the threaded rod to disengage from the implant.

17. (canceled)

18. The orthopedic cup impactor of claim 16, wherein the curved shaft comprises a planar top surface.

19. (canceled)

20. The orthopedic cup impactor of claim 16, wherein an offset radius of curvature of the curved shaft ranges from 10 cm to 20 cm.

21. (canceled)

22. (canceled)

23. The orthopedic cup impactor of claim 16, wherein the cup engagement longitudinal axis is offset with respect to the handle longitudinal axis by an offset distance from 1 cm to 10 cm.

24. (canceled)

25. The orthopedic cup impactor of claim 16, wherein the threaded rod of the drive train is at an angle from 120° to 140° with respect to an axis D-D that extends lengthwise through the drive rod of the drive train,

26.-37. (canceled)

38. An orthopedic cup impactor, which comprises:
a) a handle extending along a handle longitudinal axis from a proximal handle end to a distal handle end;
b) a curved shaft extending from a proximal curved shaft end to a distal curved shaft end, wherein the proximal curved shaft end is connected to the distal handle end;
c) a cup engagement portion extending along a cup engagement portion longitudinal axis from a distal end of the cup engagement portion to a proximal end of the cup engagement portion connected to the distal curved shaft end, wherein an apex of the curved shaft is intermediate where the proximal curved shaft end is connected to the distal handle end and where the distal curved shaft end is connected to the proximal end of the cup engagement portion;
d) a drive train at least partially disposed within the curved shaft and within the cup engagement portion, the drive train comprising:
i) a drive rod extending from a proximal drive rod end to a distal drive rod end;
ii) a proximal U-joint connected to the distal drive rod end, the proximal U-joint comprising a first pair of forwardly-extending yoke plates;
iii) an H-joint comprising opposed pairs of forwardly and rearwardly extending yoke plates;
iv) a distal U-joint comprising a rearwardly-extending pair of yoke plates, and
v) wherein the rearwardly-extending pair of yoke plates of the H-joint is pivotably connected to the forwardly-extending pair of yoke plates of the proximal U-joint and the forwardly-extending pair of yoke plates of the H-joint is pivotably connected to the rearwardly-extending pair of yoke plates of the distal U-joint; and
vi) a threaded rod extending distally from a distal end of the distal U-joint, for releasable connection to an implant; and
e) wherein the cup engagement longitudinal axis is parallel to the handle longitudinal axis, and
f) wherein an imaginary line extending perpendicularly from the cup engagement portion longitudinal axis intersects the handle longitudinal axis and then the apex such that the curved shaft apex is a greater distance from the cup engagement longitudinal axis than from the handle longitudinal axis, and
g) wherein the proximal drive rod end is adapted for releasable connection to a source of rotary motion so that rotational movement imparted to the drive train in a first direction causes the threaded rod to engage with an implant and rotational movement imparted to the drive train in a second, opposite direction causes the threaded rod to disengage from the implant.
